(19) United States
(12) Patent Application Publication
(10) Pub. No.: US 2022/0054153 A1
(43) Pub. Date: Feb. 24, 2022

(54) DEVICE AND METHOD FOR TREATMENT OF DEEP VEIN THROMBOSIS AND PULMONARY EMBOLISM

(71) Applicants: Sheri Albers, Sacramento, CA (US); John Kucharczyk, Reno, NV (US); Gregory Latchaw, Wellington, FL (US); Johannes van Vaals, Lakewood Ranch, FL (US)

(72) Inventors: Sheri Albers, Sacramento, CA (US); John Kucharczyk, Reno, NV (US); Gregory Latchaw, Wellington, FL (US); Johannes van Vaals, Lakewood Ranch, FL (US)

(21) Appl. No.: 17/460,210

(22) Filed: Aug. 28, 2021

Related U.S. Application Data
(60) Provisional application No. 63/069,008, filed on Aug. 22, 2020.
(60) Provisional application No. 63/071,546, filed on Aug. 28, 2020.
(60) Provisional application No. 63/071,597, filed on Aug. 28, 2020.
(60) Provisional application No. 63/071,633, filed on Aug. 28, 2020.

(51) Int. Cl. A61B 17/221

(52) U.S. Cl. CPC A61B 17/221; A61B 2017/22079; A61B 2017/2212

(57) ABSTRACT

An intravascular thrombus retraction device and method utilizing wires compressible into a compact form within a catheter and are self-expandable into a wire mesh web with fluid-penetrable openings in the wire mesh small enough to filter clot particles. A base of the wire mesh web is connected to a radially ring-shaped structure supporting and maintaining an opening in the base of the wire mesh and forming a thrombus capture volume. The ring-shaped structure is compressible into the catheter and is self-expandable when free of compressive forces within the catheter to open up into the open, expanded ring-shaped structure, maintaining the opening in the opening in the base of the wire mesh.

### RELATED APPLICATIONS DATA

This application claims priority under 35 U.S.C. 120 as a continuation-in-part application from U.S. Provisional Patent Applications 63/069,008, filed 28 Aug. 2020; 63/071,546, filed 28 Aug. 2020; 63/071,597; filed 4 Sep. 2020; and 63,071,633, filed 9 Sep. 2020. Each of the provisional applications was made by the same four inventors, and are incorporated by reference herein.

### BACKGROUND OF THE INVENTION

### 1. Field of the Invention

This invention pertains to intravascular medical devices for isolating, capturing, and removing blood clots from a blood vessel. This same system may also be used to retrieve obstructions, using coils, balloons, or catheter fragments dislodged during interventional procedures from the blood stream. The same system may also be used to remove obstructions from ducts and other cavities of the body, such as, for example, foreign bodies or stones from the urinary or the biliary tracts. In particular, this invention relates to medical devices for the intravascular treatment of deep vein thrombosis (DVT) and acute pulmonary embolism (PE).

### 2. Background of the Art

The present invention pertains generally to thrombus that may produce a clot in a patient's vasculature. Clots can restrict blood flow to body tissues, in which blockage or obstruction may lead to serious medical consequences, including DVT and PE. Thromboembolism occurs when a blood clot trapped within a blood vessel breaks loose and travels through the blood stream to another location in the circulatory system, resulting in an obstruction at the new location. When a clot forms in the venous circulation, it may lodge within a pulmonary blood vessel causing PE. A PE can decrease blood flow through the lungs, which in turn causes decreased oxygenation of the lungs, heart and rest of the body.

Conventional approaches to treating thromboembolism include clot reduction and/or removal. Anticoagulants can prevent additional clots from forming and thrombolytics can be partially disintegrate the clot. However, such agents typically take a prolonged period of time and in some instances can induce hemorrhage. Transcatheter clot devices can cause trauma to the vessel, are hard to navigate to the pulmonary embolism site, and may be expensive to manufacture. Surgical procedures come with increased cost, procedure time, risk of infection, higher morbidity, higher mortality, and recovery time. Accordingly, there is need for better devices and methods.

DVT and PE are considered as part of the same venous thromboembolism (VTE) disease process. The most frequent long-term complication of DVT is post-thrombotic syndrome (PTS). Veins in the leg or pelvis are most commonly affected, including the popliteal vein, femoral vein, iliac veins of the pelvis, and the inferior vena cava. Upper extremity DVT most commonly affects the subclavian, axillary, and jugular veins. Acute PE represents the most serious clinical manifestation of VTE disease. In patients with hemodynamically significant PE, systemic thrombolysis improves right ventricular dysfunction and reduces pulmonary artery pressures. However, systemic thrombolysis is associated with a risk of bleeding, particularly intracranial hemorrhage. An alternative to direct infusion into the pulmonary artery using an infusion catheter may provide the benefit of clot retraction to reduce the risk of bleeding.

Once DVT or PE has been diagnosed, treatments can range from anticoagulation alone, catheter-directed thrombolysis, full-dose systemic thrombolysis, reduced-dose systemic thrombolysis, catheter embolectomy, or surgical embolectomy. Anticoagulants can prevent additional clots from forming, and thrombolytics can dissolve the clot. However, such agents can cause hemorrhage and typically take hours or days before the treatment is effective.

Various medical devices have been used commercially in treating DVT and PE, including examples disclosed by U.S. Pat. Nos. 10,238,406, 10,524,811, 10,342,571, 10,098,651, 10,045,790, 10,588,655, 10,349,690, 10,335,186, 10,231,751, 9,844,387, 9,700,332, 9,408,620, 9,717,519, 9,439,664, and 9,427,252.

However, none of the devices currently available is ideal for treating DVT or PE. The ideal thrombectomy device would be designed to retract hard and soft clots in DVT and PE patients in a single pass without trauma to the vessel. An essential aspect of a DVT/PE thrombectomy device is its effectiveness at removing obstructive thrombi, thereby achieving a rapid improvement in hemodynamics and avoiding ischemic complications. The ideal device would allow rapid passage and advancement into veins and arteries, but must also filter distal thrombi. The device must be safe for the patient without causing damage to vascular structures, and blood loss during the procedure must be minimized. Only 3-5% of DVT and PE cases are treated today with mechanical clot removal devices. Currently, all devices for thrombectomy are costly. There is therefore an ongoing unmet need for new devices and approaches that can safely and reliably removal clots in DVT and PE patients.

### SUMMARY OF THE INVENTION

An intravascular thrombus retraction device includes wires that are compressible into a compact cylindrical form within a catheter and which are self-expandable into a wire mesh web with at least some parallel wires forming openings in the wire mesh sufficient to allow fluid passage and small enough to filter particles of at least 0.001 mm, a base of the wire mesh web connected to radial ring-shaped structure supporting and maintaining an opening in the base of the wire mesh and forming a thrombus capture volume, the radial ring-shaped structure being compressible into the catheter and being self-expandable when free of compressive forces within the catheter to open up into an open, expanded, radial ring-shaped structure which maintains the opening in the opening in the base of the wire mesh. There are also multiple intermediate guide wires connected to and spaced about the ring-shaped structure, the multiple intermediate guide wires are connected to withdrawal guidewires extending into the catheter. It is preferred that at least some of the wires in the wire mesh have protrusions extending inwardly into the thrombus capture volume, at least some of the protrusions having a height less than a distance between the at least some parallel wires.

A method for performing a thrombectomy with this device is also disclosed in which the method is executed by:
a. inserting the device into a blood vessel having a thrombus;
b. advancing a distal end of the catheter of the device towards a thrombus;
c. deploying the compressed wire mesh and ring-shaped structure distally past the thrombus;
d. expanding the wire mesh and ring-shaped structure past the thrombus;
e. retracting the wire mesh and ring-shaped structure by applying tension to the withdrawal guidewires in a first retraction step; and
f. capturing the thrombus within the wire mesh during the first retraction step.

Cardiovascular disease may arise from accumulation of atheromatous material on the inner walls of vascular lumens. If a partially or completely occluded vessel provides blood to sensitive tissue such as the brain or heart, for example, serious tissue damage may result. Vascular deposits may restrict blood flow through an artery and can cause ischemia in the heart, legs, lungs, or the brain, which may lead to pain, swelling, wounds that will not heal, amputation, stroke, myocardial infarction, and/or other conditions.

The present disclosure discloses a medical device capable of retracting DVT and PE clots from blood vessels using a collecting mechanism and aspiration, so that the retraction device and clot are withdrawn proximally through the guiding catheter out of the body. Deposits may be treated by drugs, bypass surgery and atherectomy, including a variety of catheter-based approaches based on intravascular removal of deposits occluding a blood vessel. A catheter-based system may be utilized for removing a thrombus, wherein the catheter may be extended distal to a thrombus in a blood vessel wherein the thrombus is retracted from the vessel.

A limiting factor with available thrombectomy catheter devices is the difficulty to identify and treat hard thrombus. Current thrombectomy devices do not reliably break the thrombus away from the wall of the vessel. Current thrombectomy catheters are typically bulky and require manipulation towards the thrombus to avoid the risk of distal embolism.

Basic aspiration catheters have a proximal end connected to a suction pump which causes fluid to enter the distal opening of the hollow lumen and travel to the proximal end of the lumen. Conventional aspiration catheters are typically threaded through a balloon guide catheter. In one exemplary procedure, the balloon of the guide catheter is inflated to occlude the vessel. The distal end of the aspiration catheter is typically advanced to the blood clot, with suction connected to the aspiration catheter to cause flow reversal.

One fundamental issue with thrombectomy catheters is that thrombotic burden can be highly variable. Mechanical catheters may have size constraints with respect to their use on larger thrombi. Aspiration devices have operational limits when the diameter of the catheter limits their use to small thrombi. Large thrombi on the other hand, will not pass into the catheter,; which creates a risk of embolism. Since blood is extracted alongside the thrombus in the thrombectomy procedure, aspiration can potentially cause hemodynamic deterioration in patients with pulmonary-embolism-related shock. The flexibility and durability of aspiration catheter systems may thus limit their use.

Thrombi normally must deform to the inner diameter of the aspiration catheter. The applied vacuum may partially draw a thrombus into the distal opening of the aspiration catheter's lumen, thereby deforming some of the thrombus to the catheter's inner diameter. If the thrombus becomes lodged within the distal opening of the aspiration catheter, the only option is to pull the clot back through the balloon guide. Pieces of the clot can break off during movement. When the clot is drawn out from the patient, it is difficult to confirm that the entire thrombus was removed.

An aspiration system that increases the first-pass recanalization rate can be a useful metric. Prior art systems are often not able to react quickly enough to keep the distal end of the catheter from experiencing a positive pressure. Thus, a need exists to overcome the problems with recanalization systems, designs, and processes.

One aspect of the present disclosure is to provide a mechanical thrombectomy system that is flexible enough so that it can reliably and safely navigate blood vessels to a clot.

A second aspect of the present disclosure is to provide a mechanical thrombectomy device that can reliably entrap a soft or hard thrombus without fragmenting the thrombus or damaging the intima of the blood vessel.

A third aspect of this disclosure is to provide a mechanical thrombectomy device that is biocompatible and compatible with standard medical catheters.

A fourth aspect of this disclosure is to provide a mechanical thrombectomy device that can safely and completely remove large clots of any density from the upper leg, pelvis, and lung.

A fifth aspect of the disclosure is to provide a mechanical thrombectomy device that reduces the risk of fragmentation and distal embolization when used in association with aspiration.

A sixth aspect of this disclosure is to provide an aspiration system that increases the first-pass recanalization rate during thrombus removal.

### BRIEF DESCRIPTION OF THE DRAWINGS

This invention may be more completely understood with respect to the following description of various embodiments. While the disclosure is amenable to various modifications and alternative forms, specifics have been shown by way of example in the drawings and will be described in detail.

FIG. 1A-G show serial views of the intravascular thrombectomy catheter during isolating, capturing, and removing blood clots from a blood vessel.

FIG. 1A shows a schematic view of the catheter device for retracting intravascular thrombotic material.

FIG. 1B shows the intravascular thrombectomy catheter device with its distal tip extending beyond the distal edge of the thrombus.

FIG. 1C shows the thrombectomy catheter device collapsed inside the retraction catheter with the guidewire removed.

FIG. 1D shows the thrombectomy device with catheter components deployed.

FIG. 1E shows the retraction catheter being pulled with the thrombectomy device over the thrombus.

FIG. 1F shows the collecting basket being positioned to collect the thrombus.

FIG. 1G shows the collecting basket deployed over the thrombus.

FIG. 1H is a cross-sectional illustration of a multi-lumen catheter with an inserted optical fiber that can be used to measure an optical signal in a thrombus in an artery.

FIG. 1I is a schematic of the brain of a patient illustrating a thrombus retraction procedure in which fluoroscopic images may be acquired and stored in memory by an MRI-based tracking system.

FIG. 2A-D are schematic views of the thrombectomy catheter device showing the arrangement of the struts and the ring segments in relation to a thrombus.

FIG. 3 is a schematic view of the distal embolic protection component of the thrombectomy device.

FIG. 3A summarizes studies in experimental animals.

FIG. 3B also summarizes studies in experimental animals.

FIG. 4 is a schematic of a tracking system to evaluate DVT and PE treatment in individual patients.

FIG. 4A, and FIG. 4B shows a modified clot classification system based on imaging and clot morphology.

FIG. 5 is a schematic view of the major veins in the upper leg, pelvis and thorax involved in DVT and PE.

FIG. 5B shows the use of image guidance to guide the thrombectomy device through the vasculature.

FIG. 6 is a schematic view of a custom-made handle to advance of the DVT/PE thrombectomy device.

FIG. 7 show the components of an aspiration device that can be added to the manifold attached to the hub of the guiding catheter or the collecting catheter.

FIG. 8 shows a mesh wire web having different protruding elements, protrusions, dots or gripping elements that are on the wire, generally facing inward (towards where a clot or the thrombus would be in contact with the wire.

FIG. 9 shows a predeployed 9A catheter and forward compressed wire collection element.

FIG. 9B shows the deployed 9A catheter.

FIG. 10 shows a fully deployed capture system.

FIG. 11 shows three different aspects or perspectives of the deployed ring-shaped structure's four segments.

FIG. 12 shows a different perspective of the deployed ring-shaped structure segments of FIG. 11.

FIG. 13 shows a pre-deployed 13A catheter delivery system and deployed 13B catheter delivery assembly comprising a catheter, single retraction guidewire and multiple dual wire element distal retraction guidewires.

### DETAILED DESCRIPTION OF THE INVENTION

This disclosure provides design, material, manufacturing method, and use alternatives for medical devices and systems. The device for treating DVT and PE comprises accessing a venous blood vessel of a patient in which a retraction catheter is inserted to a site of clot. An aspiration catheter with wall-mounted suction may be attached to remove a vascular obstruction with one pass. Aspiration may be applied to the guiding or collecting catheters to decrease embolization of clot fragments. An intravascular thrombus retraction device includes wires that are compressible into a compact cylindrical form within a catheter and which are self-expandable into a wire mesh web with at least some parallel wires forming openings in the wire mesh sufficient to allow fluid passage and small enough to filter particles of at least 0.001 mm, a base of the wire mesh web connected to radial ring-shaped structure supporting and maintaining an opening in the base of the wire mesh and forming a thrombus capture volume, the radial ring-shaped structure being compressible into the catheter and being self-expandable when free of compressive forces within the catheter to open up into an open, expanded, radial ring-shaped structure which maintains the opening in the opening in the base of the wire mesh. There are also multiple intermediate guide wires connected to and spaced about the ring-shaped structure, the multiple intermediate guide wires are connected to withdrawal guidewires extending into the catheter. It is preferred that at least some of the wires in the wire mesh have protrusions extending inwardly into the thrombus capture volume, at least some of the protrusions having a height less than a distance between the at least some parallel wires.

An alternative intravascular thrombus retraction device includes wires that are compressible into a compact cylindrical form within a catheter and which are self-expandable into a wire mesh web with at least some parallel or helical wires forming mesh openings in the wire mesh sufficient to allow aqueous fluid passage and small enough to filter particles of at least 0.001 mm or thrombus particles having a size which is recognized as having potentially harmful effects in at least the smaller blood vessels in the brain. A base of the wire mesh web is connected to radial ring-shaped structure supporting and maintaining an opening in the base of the wire mesh and forming a thrombus capture volume within the wire mesh. The radial ring-shaped structure could in theory be a single continuous element having an elastic memory that is the radial ring shape, but for purposes of construction of the device, a radial ring element having more than two bend or flex points, of having pivots, rotating connections, or segmented elements that allow for easier and more shapely compression may be used. The radial ring-shaped structure is as described compressible into a thin roughly cylindrical shape within the catheter and is self-expandable when free of compressive forces within the catheter to open up into an open, expanded, radial ring-shaped structure which maintains the opening in the opening in the base of the wire mesh. There are also multiple intermediate guide wires connected to and spaced about the ring-shaped structure, the multiple intermediate guide wires are connected to withdrawal guidewires extending into the catheter. It is preferred that at least some of the wires in the wire mesh have protrusions extending inwardly into the thrombus capture volume, at least some of the protrusions having a height less than a distance between the at least some parallel wires.

The wire may be a non-thrombogenic metal and the protrusions have a height of less than 0.001 mm. Because of the relatively short time duration of the device within a blood stream, there may be a tolerable range of materials that can be used if they are non-thrombogenic within the time frame of the surgery. The protrusions may be elements, bumps, rods, and the like extending from surfaces of the wires and the protrusions may have concave, convex, flat, curvilinear or pointed tips.

The device may include two catheters, a first catheter containing the wire mesh and ring-shaped structure in a compressed, non-expanded state, and a second catheter containing a compressed and expandable collection receptacle, the collection receptacle positioned within the second catheter such that upon release from the catheter, the collection receptacle expands to provide an opening in an opposed position with respect to the opening in the base of the wire mesh of a released and expanded wire mesh and ring-shaped structure.

The device may have the ring-shaped structure include or be attached to struts which place expanding or restraining force on the ring-shaped structure to maintain the opening in an expanded and open position. The device may also or alternatively have the collection receptacle is include or be attached to struts which place expanding or restraining force on the opening in the opposed position to maintain the opening in the opposed position in an expanded and open position.

A method of capturing a thrombus within vasculature may include comprising providing the above described intravascular thrombus retraction device, which may alternatively be characterized as wires that are compressible into a compact cylindrical form within a catheter and which are self-expandable into a wire mesh web with at least some parallel wires forming openings in the wire mesh sufficient to allow fluid passage and small enough to filter particles of at least 0.001 mm, a base of the wire mesh web connected to radially ring-shaped structure supporting and maintaining an opening in the base of the wire mesh and forming a thrombus capture volume, the ring-shaped structure being compressible into the catheter and being self-expandable when free of compressive forces within the catheter to open up into the open, expanded ring-shaped structure, maintaining the opening in the opening in the base of the wire mesh, multiple intermediate guide wires are connected to and spaced about the ring-shaped structure, the multiple intermediate guide wires are connected to withdrawal guidewires extending into the catheter, at least some of the wires in the wire mesh having protrusions extending inwardly into the thrombus capture volume, at least some of the protrusions having a height less than a distance between the at least some parallel wires; the method comprising:

a) inserting the device into a blood vessel having a thrombus;
b) advancing a distal end of the catheter of the device towards a thrombus;
c) deploying the compressed wire mesh and ring-shaped structure distally past the thrombus;
d) expanding the wire mesh and ring-shaped structure past the thrombus;
e) retracting the wire mesh and ring-shaped structure by applying tension to the withdrawal guidewires in a first retraction step; and
f) capturing the thrombus within the wire mesh during the first retraction step.

The wire may be composed of a non-thrombogenic metal and the protrusions have a height of less than 0.001 mm, and during the first retraction step, the protrusions engage and grasp a surface of the thrombus.

The present disclosure further relates to a method of treating DVT and PE in the peripheral vasculature of a patient. The method includes providing a thrombectomy device that can be tubular and is formed of a braided filament mesh structure. The mesh structure can have a proximal end of the attached to a distal end. The invention includes advancing a catheter with the thrombectomy device through a vascular thrombus in a venous vessel. A shaft extends through the catheter and a distal end is coupled to a proximal end. The method includes deploying the thrombectomy device from the catheter from a constrained configuration to an expanded configuration. In some embodiments, the thrombectomy device engages at least a wall of the venous vessel distally past the thrombus at full expansion. The method includes retracting the thrombectomy device proximally to separate a portion of the thrombus from the venous vessel wall while the mesh structure captures the thrombus. The method includes withdrawing the thrombectomy device from the patient to remove the thrombus from the venous vessel.

Advancing the thrombectomy device includes inserting the catheter into the venous vessel until a radiopaque distal tip of the catheter is distally past the thrombus. In some embodiments, deploying the thrombectomy device from the constrained configuration to the expanded configuration includes advancing the shaft distally until the thrombectomy device is beyond a distal end of the catheter. Deploying the thrombectomy device further includes determining a position of the thrombectomy device with respect to the catheter via imaging of a first radiopaque marker located on the catheter and a second radiopaque marker located on at least one of the shaft or mesh structure.

The vascular thrombectomy device is added into the mesh structure by entering the expandable tubular portion via at least an aperture located at the proximal end of the self-expanding stent. The method includes inserting the catheter into the venous vessel through an access site, which is a popliteal venous site, a femoral venous site, or an internal jugular venous site. The venous vessel has a diameter of at least 5 millimeters and may include a femoral vein, an iliac vein, a popliteal vein, a posterior tibial vein, an anterior tibial vein, or a peroneal vein.

The method further includes: percutaneously accessing the venous vessel of the patient with an introducer sheath through an access site into the venous vessel of the patient; advancing a distal end of the introducer sheath to a position proximal of the thrombus; inserting the catheter through a lumen of the introducer sheath so that a distal tip of the catheter is distally past the thrombus.

Withdrawing the thrombectomy device from the patient includes: retracting the thrombus extraction device relative to the introducer sheath until an opening is within the self-expanding stent; collapsing the stent portion and mesh structure so as to compress the thrombus; retracting the stent portion and mesh structure into the introducer sheath; and removing the thrombectomy device from the introducer sheath.

The method may further includes extruding at least some of the thrombus through the distal portion of the expandable tubular portion and capturing a part of the thrombus in the self-expanding funnel or further compressing the thrombus through a mesh of the self-expanding funnel. The method may further includes aspirating the thrombus through an aspiration port connected to a proximal end of the introducer sheath.

One aspect of the present disclosure relates to a method of treating DVT in a peripheral vasculature of a patient to include percutaneously accessing a venous vessel of a patient with an introducer sheath through a popliteal vein site; and inserting a catheter with a thrombectomy device through a lumen of the introducer sheath so that the catheter is distally past the thrombus.

In some embodiments of the invention, a proximal end of the mesh structure may be attached to a distal end of the fenestrated structure. The thrombectomy device may be deployed from a constrained configuration to an expanded configuration by advancing a shaft distally until the stent portion of the thrombectomy device is beyond the distal end of the catheter.

One aspect of the present invention relates to a removal of thrombus from an artery or a vein of a patient by providing a thrombectomy device with a net-like filament mesh structure; advancing with the thrombectomy device through a thrombus, and deploying the thrombectomy device to engage a wall of the blood vessel. Retracting the thrombectomy device to separate a portion of the thrombus from the vessel wall and to capture the portion of the thrombus within the net-like mesh structure to remove thrombus from the patient.

In the method of the invention, fluoroscopically monitoring deployment of the thrombectomy device beyond first radiopaque marker located on the catheter relative to a second radiopaque marker located on the thrombectomy device. In some embodiments, the thrombus is located in the peripheral vasculature of the patient and the blood vessel has a diameter of at least 5 millimeters and includes at least one of a femoral vein, an iliac vein, a popliteal vein, a posterior tibial vein, an anterior tibial vein, or a peroneal vein. In some embodiments of the invention, the method includes aspirating or infusing a thrombolytic agent into or from the blood vessel before, during, or after thrombus extraction.

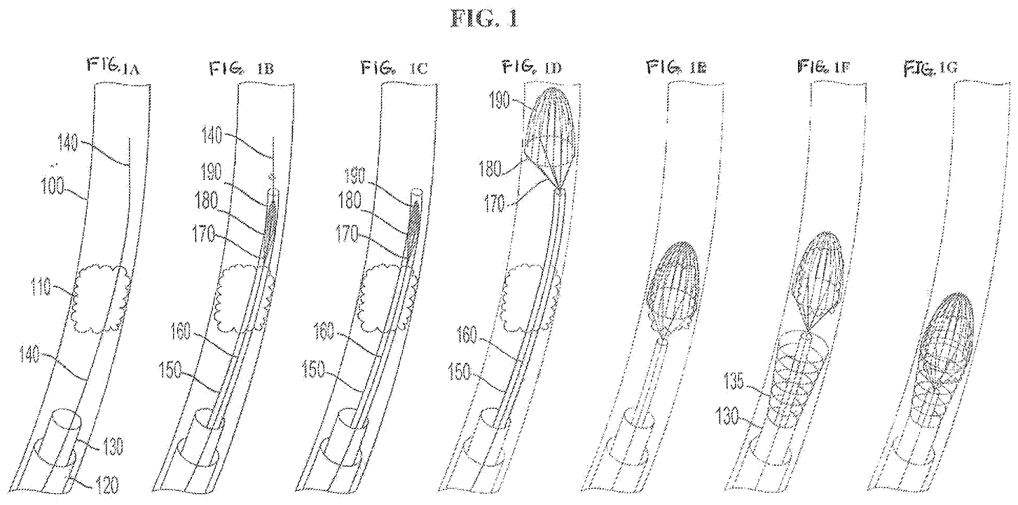

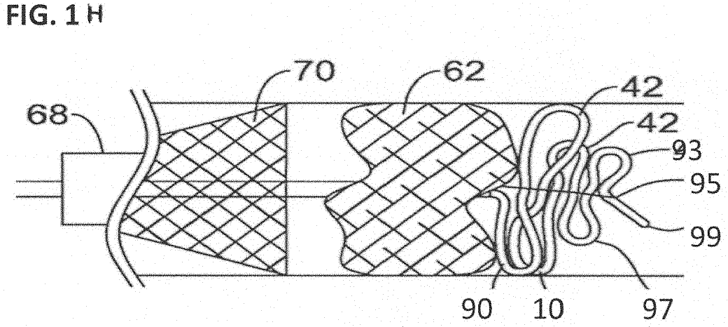

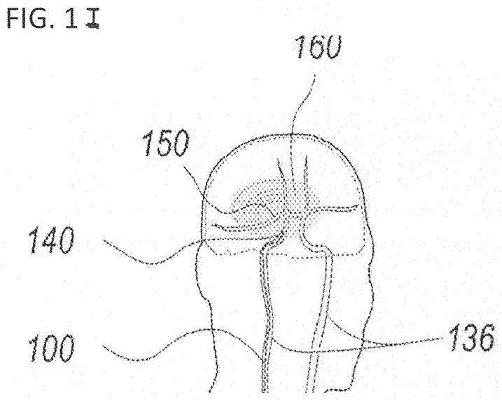

FIGS. 1A-G depict steps for the mechanical thrombectomy device in a blood vessel 100 with a thrombus 110. A guiding catheter 120 can be positioned by transluminal catheter delivery within the lumen of the blood vessel 100 proximal to the thrombus using image-guided techniques. A retraction catheter 130 can pass through the guiding catheter 120 and may be positioned just below the proximal aspect of clot 110. As shown in FIG. 1A, a guidewire 140 can be placed proximal or distal to the thrombus 110 to be used to guide the collecting or retraction catheter 130. The retraction catheter 130 can then pass through guiding catheter 120 and over guidewire 140 to a position with its distal tip placed distal to the distal edge of the thrombus 110 as shown in FIG. 1B. The thrombectomy device with retracting wire 140, struts 170, ring structure 180 and web 190 can be passed through the retraction catheter or may be pre-loaded inside the retraction catheter 130. As shown in FIG. 1C, the guidewire 160 can be removed. The thrombectomy device can be deployed by manipulating the retraction wire 160 as shown in FIG. 1D. As shown in FIG. 1E, the retraction catheter 150 can be positioned proximally with the thrombectomy device pulled down over thrombus 110 in FIG. 1A. As shown in FIG. 1F, the collecting basket 135 may be deployed from the collecting catheter 130 to collect the thrombus as shown in FIG. 1G.

When possible, the entire thrombus 110 may be pulled into the guiding catheter 120 and removed from the body, leaving the guiding catheter 120 in place. If the clot is too large to be pulled into and through the guiding catheter 120, aspiration may be applied to the guiding catheter or the collecting catheter 130, wherein a catheter connected to an aspiration system can be hooked to the flushing system for the guiding catheter via a 3-way stopcock. Aspiration can be usefully added when applied to the clot that has been pulled into the collecting catheter 140 to make it smaller for removal through the guiding catheter 120.

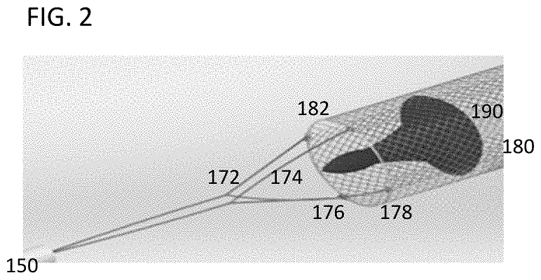

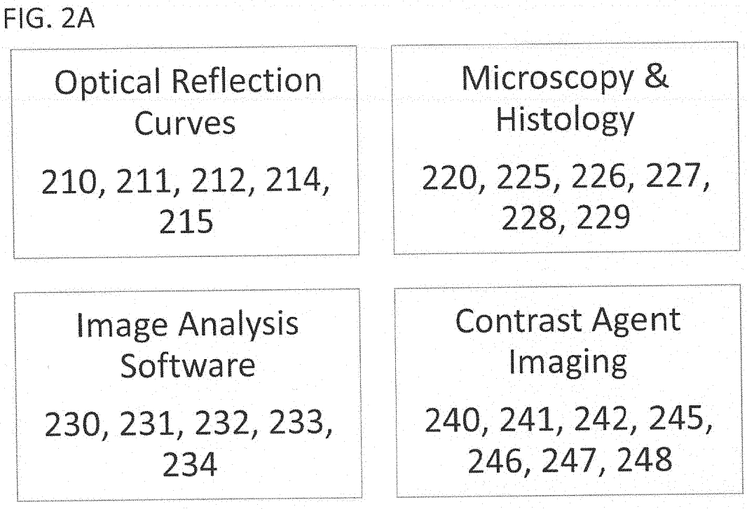

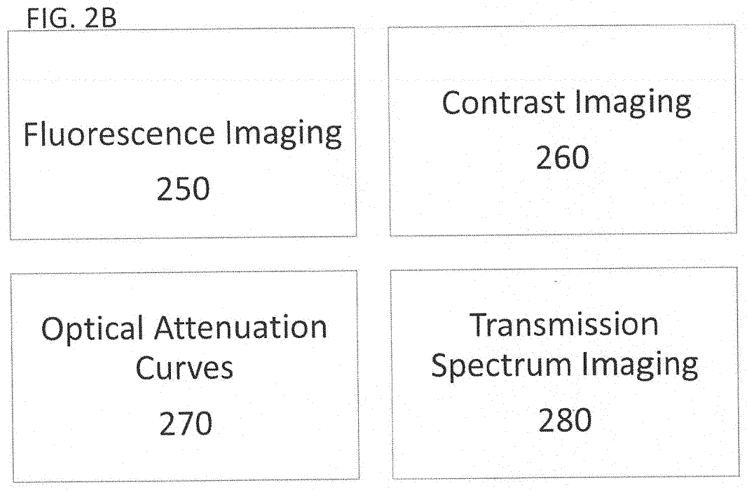

FIG. 2 is a schematic view of the thrombectomy device showing the arrangement of the struts 172, 174, 176, 178 and the ring segment 182. The device uses a web structure with retraction wires to retract thrombus 190. The device includes expandable struts having a closed compact configuration 184 and an open expanded configuration 186. In an optional embodiment, additional collection features such as cactus-claws, end-hooks and hook-type imaging may be included. In some embodiments, these features can be formed from various metals or alloys such as Nitinol, platinum, cobalt-chrome alloys, 35N LT, Elgiloy™, stainless steel, tungsten or titanium.

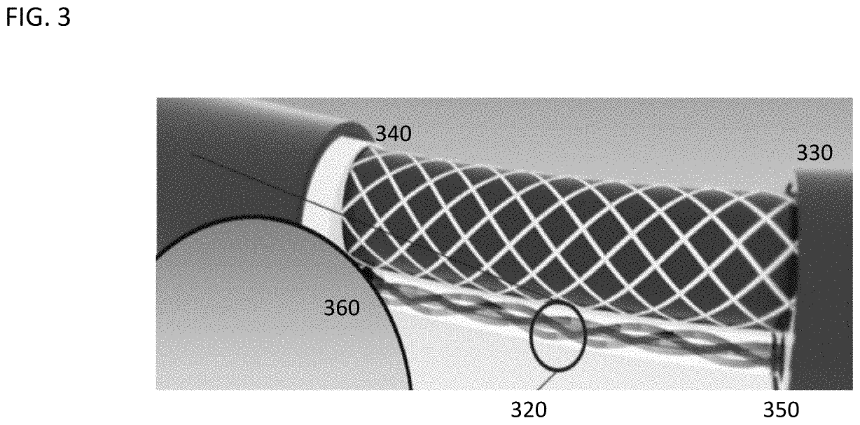

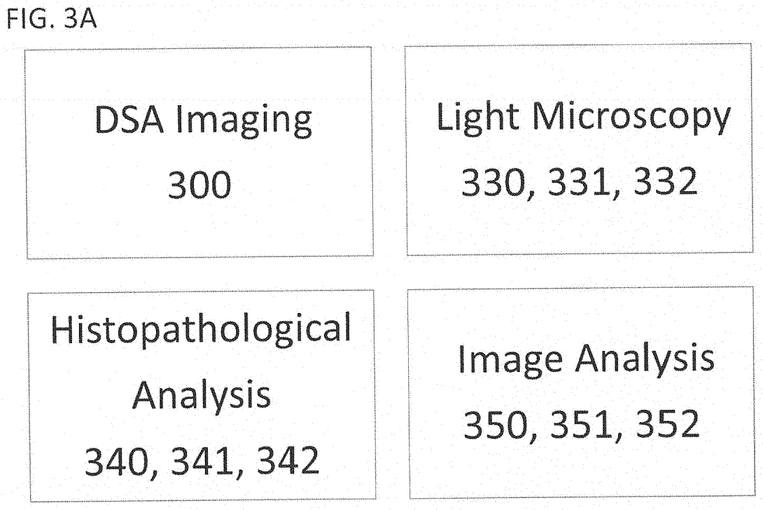

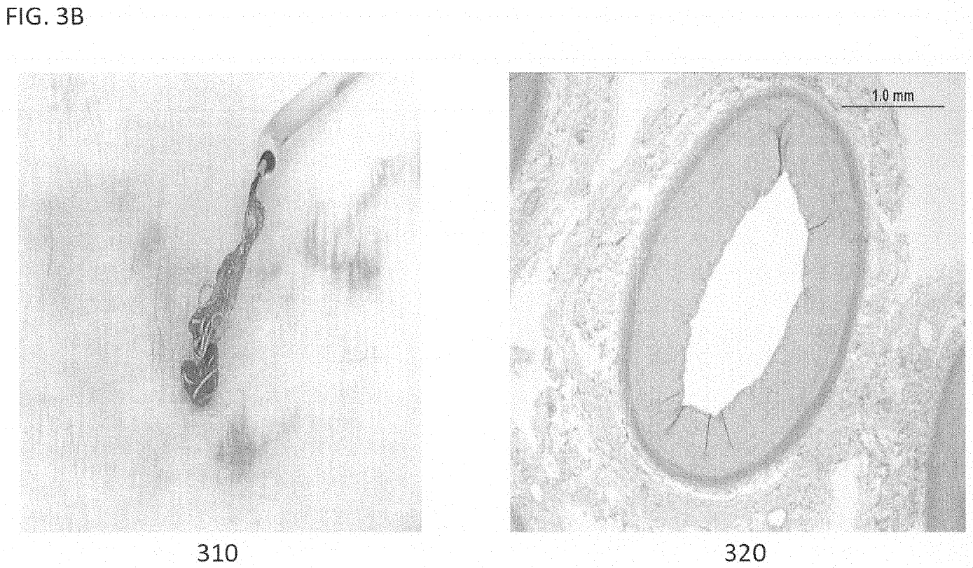

In one embodiment of the disclosure shown in FIG. 3, fluoroscopically visible markers 188 are applied to the retraction ring, the retraction wire and the collecting basket 180 and to the tip of the guiding catheter 150 to facilitate localization of all components. Some examples of radiopaque materials include gold, platinum, palladium, tantalum, tungsten alloy, and polymer material loaded with a radiopaque filler. The thrombectomy device may also be made from a metal, metal alloy, polymer, a metal-polymer composite, ceramics, or other suitable material including, such as 304V, 304L, and 316LV stainless steel; mild steel; nickel-titanium alloy such as linear-elastic and/or super-elastic nitinol; and nickel-chromium-molybdenum alloys.

In another embodiment, the device can be equipped with imaging sensors 350, or with sensors measuring physiological parameters 360 such as pressure, temperature and oximetry. In one embodiment, FOSS technology facilitates the visualization of thrombectomy catheters 330 and wires 340 without the need for fluoroscopy. In addition to reducing the need for X-ray exposure of the patient and medical personnel, the FOSS technology also enables more detailed views of device positioning. In an exemplary embodiment, optical fibers are embedded in the device and equipped with Fiber Bragg Gratings, which enables the determination in 3 dimensions of the shape and position of the catheters and wires in real-time and with high accuracy. The shape and position of the catheters and wires can then be superimposed on roadmap views of the vasculature and pathology.

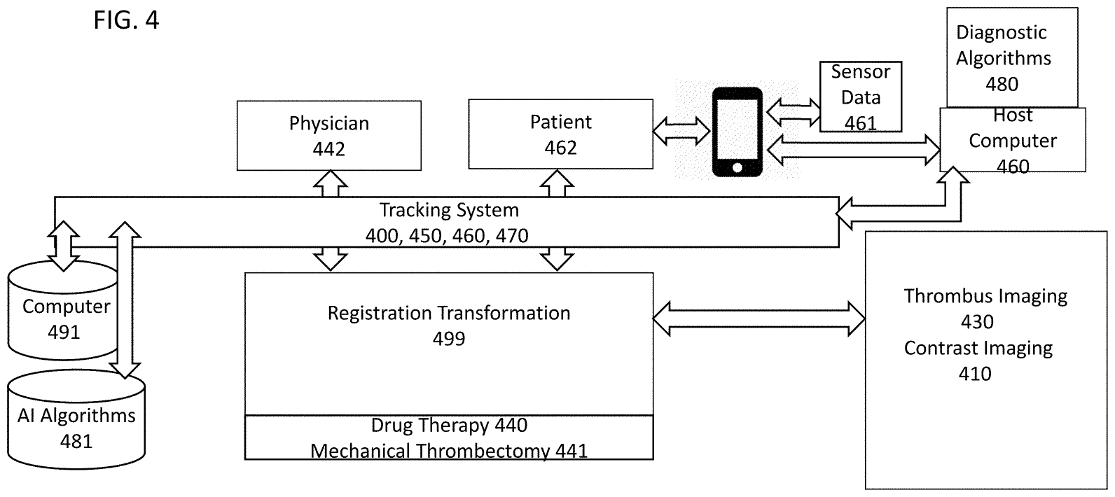

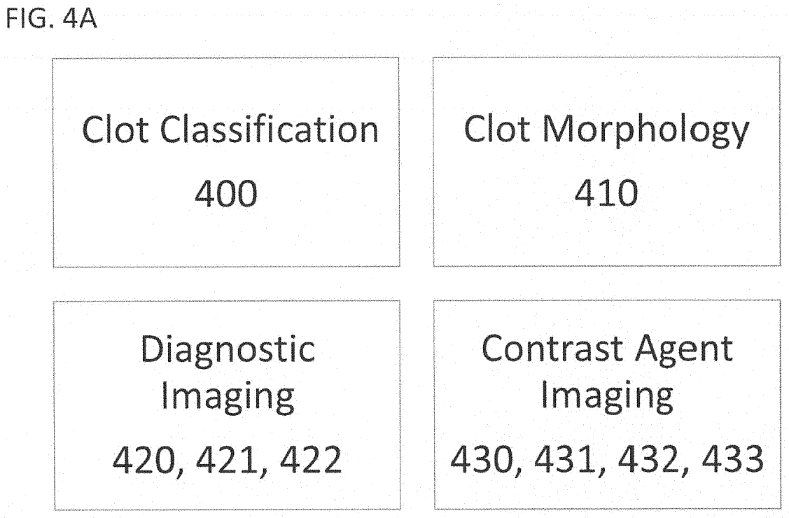

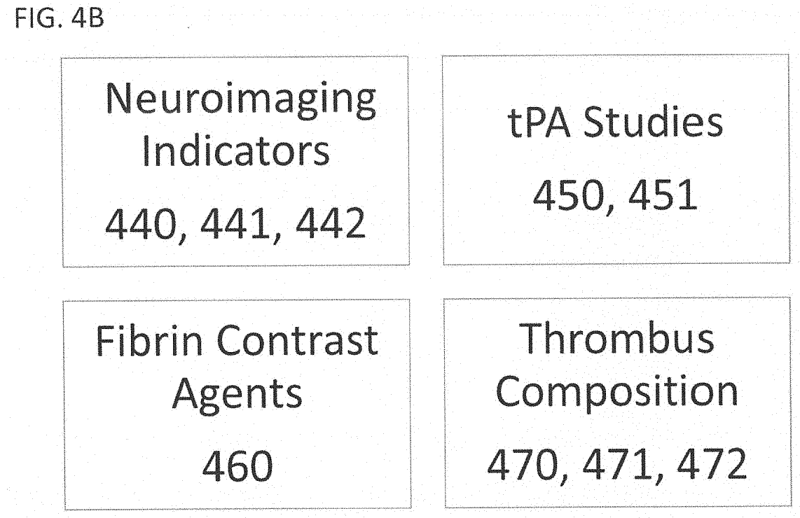

As shown in FIG. 4, sensors 461 may be connected to a host computer 461, wherein diagnostic algorithms 480 can be used to evaluate treatment plans for individual patients 462 and to actively modify existing treatment plans by physicians 442. A computer-based 491 tracking system 400 can be used in DVT and PE patient studies to investigate clot composition with respect to the age 450, composition 460 and size of a thrombus 470. During formation of a thrombus, characteristic alterations in fluorescence contrast imaging 410 and thrombus imaging 430 may be registered by the host computer 499. Observation of imaging changes 499 can be clinically useful in evaluating the potential utility of various alternative therapeutic interventions, such as, for example, drug thrombolytic therapy 440 and mechanical thrombectomy 441. Imaging 410, 430 may be used to guide the thrombectomy device through the vasculature 510 through under manual control of a host computer 460. Since branching of the main pulmonary arteries may be anatomically complex, it may be difficult to locate and catheterize an occluded vessel 499 when using single plane fluoroscopy 410.

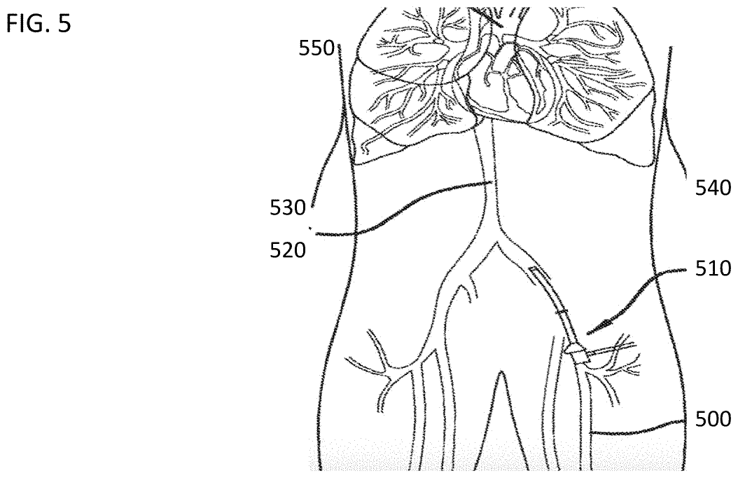

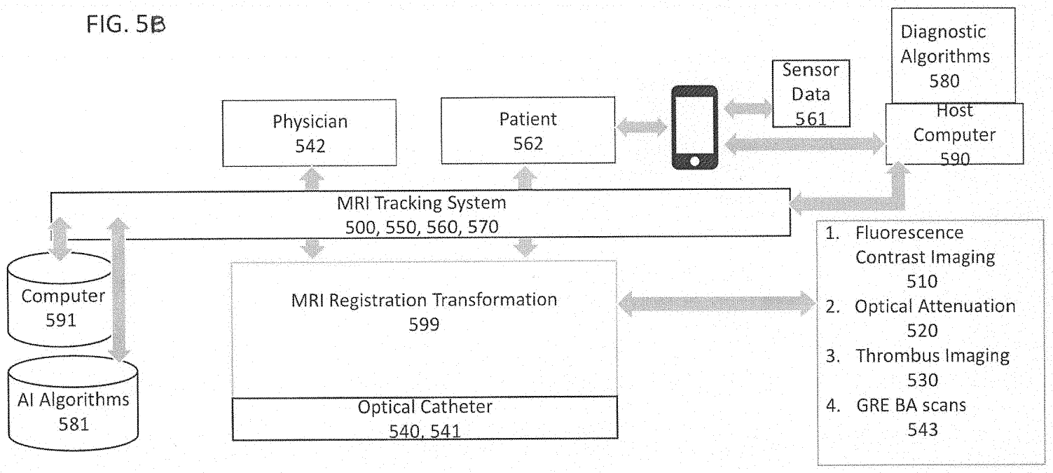

As shown in FIG. 5B, image guidance can be used to guide the thrombectomy device through the vasculature 500 toward the correct location 530, 540, to manipulate various functions of the device 510, and to manage the retraction of the thrombus 520. PE is considered to be part of the same continuum of disease as DVT with over 95% of emboli originating in the legs. Mechanical clot removal, as disclosed in the present disclosure, is relatively rapid compared to the use of thrombolytics. Pulmonary emboli, particularly those in the proximal aspects of the pulmonary arteries, can be quite large requiring a larger retriever catheter and wires than for most DVT cases. Biplane fluoroscopic systems such as those used for cerebral angiography and intervention can be used to improve the catheterization process.

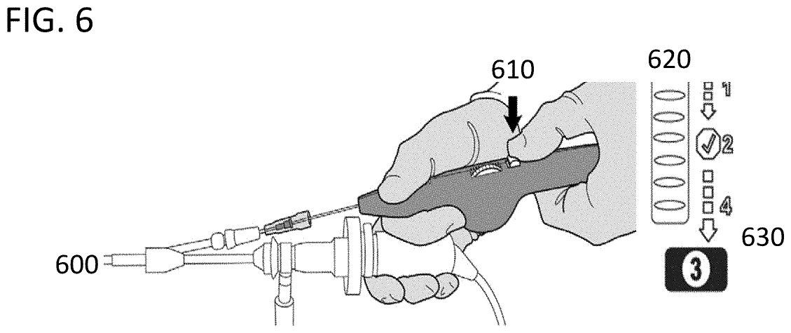

The handle 600 (FIG. 6) may have a proximal end portion and a distal end portion. The distal end portion of the handle which may be connected or attached to the proximal shaft. In some embodiments, an adaptor may facilitate a connection between the handle and the proximal shaft. The handle may be formed from a polymer material, a metal material, a combination of metal material and a polymer material, and/or one or other suitable materials. Further, the handle may be formed with a suitable forming technique including machining, molding, grinding, injection molding, and laser cutting.

In one embodiment shown in FIG. 6, the device has a custom-made handle 600, in which an operator can engage a thumb engaging surface of the device 610 to transmit movement to the device through a compressed coil spring 620, wherein the operator is able to apply a calibrated force 630 to the device. The calibrated forces may be spaced a predetermined linear distance, to include detents, cut-outs, recesses, spacings, notches, indents, bumps, protrusions and/or other features. As shown in FIG. 6, the calibration forces may be linearly drawn as it is moved one or more predetermined distances in the longitudinal direction. In one example, the adjustment member may include a portion having a protrusion to include a cut-out, recess, spacing, notch, indent, and/or other formations to facilitate engaging the restrictions in or on the handle.

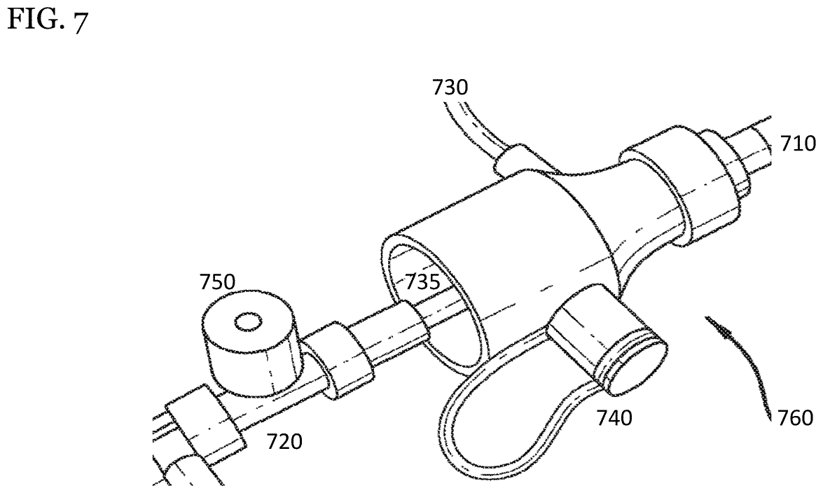

As shown in FIG. 7, a catheter 710 connected to an aspiration device 720 can be added to the manifold attached to the hub of the guiding catheter 730 or the collecting catheter 735 so that aspiration can be applied to the entire system to facilitate thrombus retraction, and prevent fragmentation and embolization of fragments through the guiding catheter 730. Aspiration coupled with mechanical thrombectomy 740 can thereby assist in the retraction of large clots. An aspirational system attached to the manifold of the guiding catheter may help reduce the size of the clot within the collecting device 735 to facilitate removal of the thrombus. Aspiration can also reduce the potential for distal embolization as the clot is manipulated.

The entire thrombus may be pulled into the guiding catheter 120 and removed from the body, leaving the guiding catheter 120 in place. If the clot 110 is too large to be pulled into and through the guiding catheter 120, aspiration may be applied to the guiding catheter or the collecting catheter 130, wherein a catheter connected to an aspiration system can be hooked to the flushing system for the guiding catheter via a 3-way stopcock 750. Aspiration 760 can thus be usefully added when applied to the clot that has been pulled into the collecting device 4 to make it smaller for removal through the guiding catheter 120.

A method of treating deep vein thrombosis and pulmonary embolisms may include accessing a venous vessel of a patient, wherein a retraction catheter containing a clot treatment device is inserted into the venous circulatory system to a site of clot, wherein an aspiration catheter in inserted with wall-mounted suction attached to its inflow port, wherein the aspiration component can remove clot and other debris, and, wherein complete removal of both soft and hard components of a vascular obstruction is completed with one pass within in ninety percent of cases.

A device that may be used in the method may include a device equipped with a collecting mechanism in the form of a collecting catheter that passes over the retraction catheter, and that is equipped with a collecting structure that can be deployed when moving the collecting catheter beyond the end of the guiding catheter, surrounding the object when the object is extracted using the retraction catheter.

The method may further include accessing a venous vessel, inserting into retraction catheter into vessel, and restoring blood flow using the clot retraction device.

An alternative multi-lumen, multi-functional catheter system may include a plurality of axial lumens, wherein at least one physiological measuring device is present within a clot retraction catheter, wherein said physiological measuring device is connected to a host computer which is equipped for receiving information regarding DVT and PE treatment plans, wherein the host computer contains a treatment planning and therapy algorithm for individual DVT and PE patients, and, wherein the host computer signals the operator to actively modify the existing treatment plan as the therapy algorithm progresses.

A thrombectomy catheter comprising: an elongate flexible catheter body having a proximal end, a distal end and a central lumen extending longitudinally through the catheter body, wherein the catheter comprises a catheter with a variable durometer outer jacket, wherein the catheter wall thickness ratio of the inner diameter to the outer diameter is 0.80 or higher, wherein the tensile strength of the catheter is higher than 2 lbs.

Another device for removing blood clots may include an intravascular catheter having a distal end and a proximal end, the catheter having an inner lumen and an outer lumen, wherein an aspiration pump is attached to the proximal end of the catheter, and a mechanically actuated positive displacement powered by a rotating motor, wherein the motor rotates at a speed below 2000 RPM when driving the aspiration pump and wherein the speed of the motor is cycled at a frequency below 10 Hz.

Another method of treating deep vein thrombosis in a peripheral vasculature of a patient may include: percutaneously accessing a venous vessel of a patient with an introducer sheath through an access site into the venous vessel of the patient; inserting a catheter constraining a thrombectomy device through the lumen of the introducer sheath so that a distal tip of the catheter is distally past a portion of the thrombus; deploying the thrombectomy device from a constrained configuration to an expanded configuration, wherein the thrombectomy device is in an expanded state between about 20 degrees and about 50 degrees; and, removing the thrombectomy device from the patient.

It will be apparent to those skilled in the art that various modifications and variations can be made in the disclosed systems and processes. It is intended that the specification and examples be considered as exemplary only.

Thrombus in the vasculature includes a range of morphologies and consistencies. Typically, older thrombus material contains a higher percentage of fibrin, making it less compressible with a harder outer surface that makes it more difficult to ensnare or aspirate than more acute thrombus which is softer. Current mechanical thrombectomy devices may not penetrate the surface of a hard fibrin-rich thrombus or produce sufficient force to grip the thrombus. It can be very difficult to aspirate a hard thrombus without first breaking it into pieces, which could then embolize into distal branches. During thrombectomy, 75-85% of thrombi can be removed using current devices, such as stent-retrievers and aspirators. However, the remaining 15-25% of intravascular thrombus cannot be easily removed by mechanical devices because the thrombus is hard.

CT and fluoroscopy imaging cannot typically identify the composition of intravascular thrombus, which may vary from relatively hard to relatively gel-like and soft. An obstructing thrombus in a blood vessel of the brain can be a medical emergency caused by occlusion of blood vessels to the brain or within the brain. Although an ischemic event can occur anywhere in the vascular system, the carotid artery bifurcation and the origin of the internal carotid artery are the most frequent sites for thrombotic occlusions of cerebral blood vessels.

Methods for imaging thrombus are reviewed in the present disclosure. As used herein, an imaging technology may include, positron-emission tomography, single photon emission computed tomography, magnetic resonance imaging, optical imaging, ultrasound, photoacoustic imaging, computed tomography, or near-infrared fluorescence-imaging. FIG. 1H is a cross-sectional view of a catheter 10 in an artery adjacent to intravascular thrombus 62. In an exemplary embodiment of the disclosure, an optical fiber 95 is used to measure an optical signal collected from the thrombus 62, which is then output to an MRI-based tracking system 95. The catheter 10 may be advanced to the thrombus 62 to illuminate the thrombus 62, wherein an optical measurement unit 70 enables identification of the optical signal of the thrombus 42 which reflects the content of the thrombus 62. A detector in the MRI system 99 converts the optical signal 42 into an electrical analog signal, and an analog-to-digital conversion circuit 93 digitizes the signal to the MR tracking system 95 for analysis.

In one embodiment of the disclosure shown in FIG. 1I, a schematic of the brain of a patient illustrates a thrombus retraction procedure 100 in which fluoroscopic images 136, 150, 160 may be acquired and stored in memory in a host computer for subsequent retrieval by an MRI-based tracking system 140.

FIG. 2A shows optical reflection curves 210 that summarize in vitro studies to evaluate hard clot 212, gel-like clot 213 and soft clot 214 collected in vitro. The partial thromboplastic time coagulation assay 215 was used in pig blood in glass test_tubes. Thrombi were placed on gauze and photographed 220 based on linear thrombus dimensions. Thrombus was formalin-fixed, embedded in paraffin, and stained with hematoxylin and eosin. Histological sections 225 were photographed based on erythrocyte-rich 226 and platelet-fibrin 227 accumulations, and neutrophil 228 and monocyte 229 deposits.

In one embodiment, contrast agents were used for optical imaging 230, fluorescence 231, luminescence 232 or acousto-optical imaging 233. In one example, silicon containing nanoparticles 234 were used to produce fluorescence and luminescence signal. Other contrast agents can include nanospheres 240, such metal oxide nanoparticles 241, and quantum dots 242. Photoacoustic imaging contrast agents can include photoacoustic imaging-compatible agents 245, such as methylene blue 246, single-walled carbon nanotubes 247, and gold nanoparticles 248. FIG. 2B illustrates another embodiment in which thrombi were formed from platelet poor plasma in well plates with phosphate-buffered saline. A non-specific binder was added to the thrombi for 30 minutes then the thrombi were washed with PBS prior to fluorescence imaging 250. Thrombi were subsequently washed and then fluorescence imaged again. Thrombus was imaged by adding an imaging agent to a cell culture or tissue culture containing a thrombus to generate a detectable signal within a fluorescence image 260.

In another embodiment, the optical attenuation characteristics of hard thrombus were evaluated compared to gel-like and soft thrombus. The ratio of reflected to incident light intensities of optical attenuation curves 270 was used to determine whether the thrombus is hard or soft. In another embodiment, the transmission spectrum of light 280 produced characteristic wavelengths due to absorption by oxygenated red blood cells was compared to a hard thrombus and soft or gel-like clots to determine the presence of deoxygenated hemoglobin in the clot.

FIG. 3A, and FIG. 3B summarizes studies in experimental animals. Digital subtraction angiography 300 was used to confirm vascular occlusion in thrombectomy studies. Photographs of retracted thrombotic material were compared with the architecture of the retained clot 310. Clots were placed on gauze and photographed, gross measurements of linear thrombus dimensions were taken, formalin-fixed, then embedded in paraffin and stained with hematoxylin and eosin. As shown in FIG. 3B, histological sections of carotid artery 320 were photographed after thrombus removal 310.

In one embodiment, clot composition was categorized by light microscopy as RBC-dominant 330, fibrin-dominant 331, or mixed 332. Histopathologic analysis included quantitative and qualitative measurements for RBC 340, WBC 341, and fibrin 342. Image analysis software was used to measure quantities of fibrin 350, RBCs 351, and WBCs 352.

FIG. 4A, B shows a modified clot classification system based on imaging and clot morphology. Both HMCAS 421 and BA 422 were significantly associated with the presence of red blood cell-dominant clots 410.

In another embodiment, magnetic resonance imaging contrast agents included a chelating agent selected from paramagnetic metal ions 430, such as Gd(III) 431, Dy(III) 432 and Fe(III) 433.

In another embodiment of the present study, neuroimaging indicators 440 were used to distinguish “red thrombi” 441 from “white thrombi” 442.

In another embodiment, stroke resulting from erythrocyte-rich thrombus 451 in the venous system were evaluated after treatment with recombinant tissue plasminogen activator 450.

In another embodiment, fibrin-specific MRI contrast agents 460 were evaluated for identification of thrombus composition 470 to establish the clot size 471 and composition 472 before and following thrombectomy.

FIG. 5B is a schematic of an MRI-based tracking system 500 which can be used in patient stroke studies to investigate clot composition with respect to the age 550, composition 560 and size of a thrombus 570. During formation of a thrombus, the concentration of paramagnetic hemoglobin and methemoglobin within the clot changes resulting in characteristic alterations in fluorescence contrast imaging 510, optical attenuation 520 and thrombus imaging 530. Observation of these MR imaging changes 599 can be clinically useful in evaluating the potential utility of various alternative interventions, such as, for example, drug thrombolytic therapy 540 and mechanical thrombectomy 541 used by the physician 542. Other changes can also be monitored by diagnostic algorithms 580 and AI algorithms 581 by the host computer 590 and other computers 591.

In one embodiment of the present disclosure, pathological changes in thrombi can be evaluated in terms of clot composition 510, 520, 530. Noninvasive imaging can be acquired in acute stroke cases with non-contrast CT or MRI protocols including gradient-recalled echo sequences 570. Comparisons in stroke patients can include acute cerebral occlusion with non-contrast CT or GRE sequences acquired immediately before endovascular thrombectomy using an optical catheter 540, 541 blinded to clinical, angiographic and pathological variables.

A method for visualizing thrombus in an artery includes a wavelength-specific reflector being advanced to traverse the thrombus, wherein the incident light is selectively reflected at the diagnostic wavelength after interacting with the thrombus, wherein passing the optical signal through the thrombus increases an optical attenuation signal compared with a single pass, wherein the host computer analyzes transmitted optical signals, and, wherein the host computer identifies whether the thrombus is hard or soft based on the wavelength signal.

In the method, an optical fiber is adapted to allow light to interact with the thrombus, wherein hard thrombus absorbs less light than thrombus, and, wherein the MRI system can establish the composition of the thrombus based on the optical attenuation of the thrombus.

A device for tracking thrombus in a patient's vasculature may include a measuring device connected to a host computer that can evaluate thrombus retraction, wherein the device is equipped with both optical sensors and imaging sensors, wherein the host computer contains a therapy algorithm for individual patients, and wherein the host computer can actively modify thrombus retraction as the therapy algorithm progresses.

The above device may have the host computer determine the thrombus composition based its optical transmission, and the MRI can be used to evaluate whether thrombus composition reduces its susceptibility to recombinant tissue plasminogen activator. The host computer may determine retraction routes, speed and status of thrombus for individual patients.

A method for tracking thrombus in the vasculature comprising analyzing the intensity of an optical signal from a sensor in a catheter positioned in a blood vessel of a patient may include using an optical signal from the sensor is attenuated by thrombus, wherein an MRI-based host computer tracks the thrombus by analyzing the measured signal attenuation, and, wherein the location of the thrombus is converted by the MRI-bases host computer into MRI coordinates using a registration transformation.

It should be understood that the foregoing description is merely illustrative of the present disclosure. Various alternatives and modifications can be devised by those skilled in the art without departing from the scope or spirit of the disclosure.

A further method of treating deep vein thrombosis in a peripheral vasculature of a patient, the method comprising: percutaneously accessing a venous vessel of a patient with an introducer sheath through an access site into the venous vessel of the patient; inserting a catheter constraining a thrombectomy device through the lumen of the introducer sheath so that a distal tip of the catheter is distally past a portion of the thrombus; deploying the thrombectomy device from a constrained configuration to an expanded configuration, wherein the thrombectomy device is in an expanded state between about 20 degrees and about 50 degrees; and, removing the thrombectomy device from the patient.

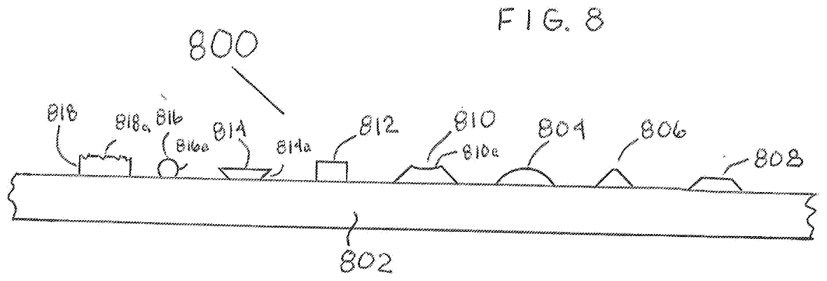

FIG. 8 shows a mesh web wire system 800 having different protruding elements. protrusions, dots, or gripping elements (804, 806, 808, 810, 812, 814, 816 818) that are on the wire 802, generally facing inward (towards where a clot or a thrombus would be in contact with the wire 802. Different structures for these protruding elements are exemplified separately to show how the different shapes and dimensions and configurations can be selected to provide unique and designed functions with respect to different types of clots, thrombus and debris based on size, texture, rheology and dimensions of the unwanted materials to be captured. Protrusion 804 is a generic and simplest protrusion to form and likely the easiest to manufacture, comprising a truncated spherical dot.

Element 806 is a pyramidal element, with a more pointed tip to grasp thrombus with texture and hard surfaces. Element 808 has inwardly sloped side rising to a flat surface to grasp softer clots. Element 810 has inwardly sloped side rising to a concave surface 810a to grasp soft clots and with edges of the concave surface grasping into the clot, yet retaining a large surface area of contact with the particle to be removed. Element 812 is again a relatively generic and simple protrusion to form. Element 814 is shown with outwardly sloped sides 814a which can be used to trap smaller particles as the wire mesh is withdrawn, the sloped sides capturing particles that might even escape the wire mesh. Element 816 is a truncated spherical element, with the cut through the sphere sufficiently low as to again create inwardly sloped surfaces 816a which may provide the small particle capture function described for element 814 above. Element 818 is shown with a textured surface 818a which can assist in grasping clots that might have smoother or more slippery surfaces.

A textured, grooved, irregular surface such as in 818a can be provided on any of the individual structures. Many techniques for forming such surfaces such as embossing, leaching of soluble materials (e.g., soluble polymers, salts, sugars, etc.) in the deposited metal, ceramic, composite or polymeric elements, and the like.

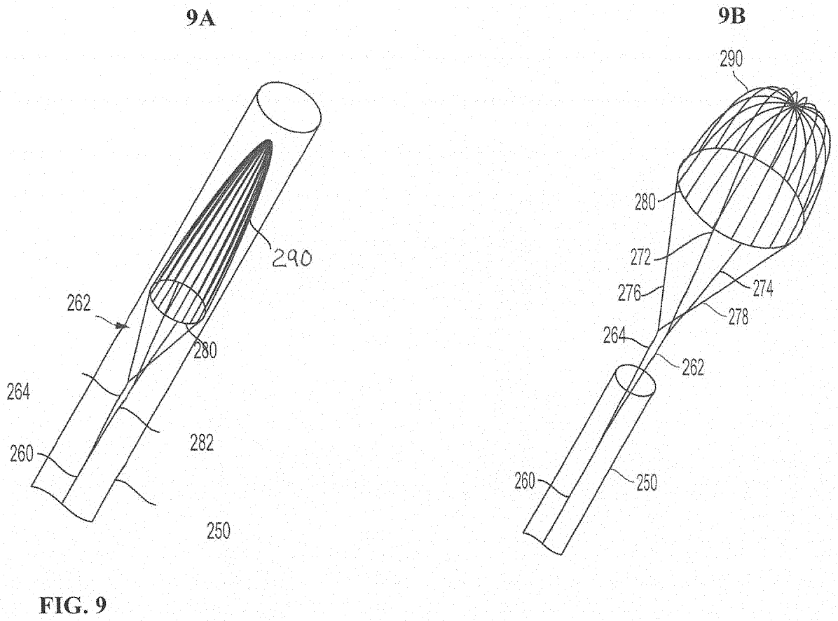

FIG. 9 shows a predeployed 9A catheter 250, forward compressed wire collection element 290 (cross-web wires not shown), compressed ring-shaped element 280, distal guidewires 262 connected to the ringshaped element 280, primary withdrawal guidewires 264, 282, and the external lead guidewire 260. FIG. 9B shows the deployed system with the distal guidewire set 162 of 9A shown as deployed individual distal guidewires 272, 274, 276, 278.

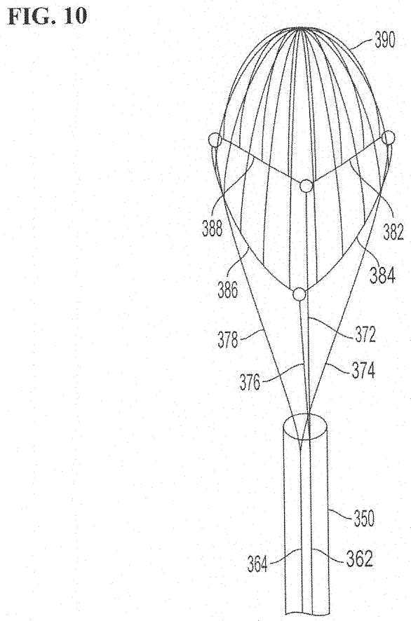

FIG. 10 shows a fully deployed capture system (with the crosswires in the capture web 390 not shown to facilitate other distinguishing elements. The circular ring-shaped element is circular in an aspect view, but from this side view, the connection points for the distal guidewires 372, 374, 376, 378 to the segments of the ring-shaped structure 382, 384, 386, 388 are not within a single plane. The primary retraction guidewires 362, 364 are shown still within the deployment catheter.

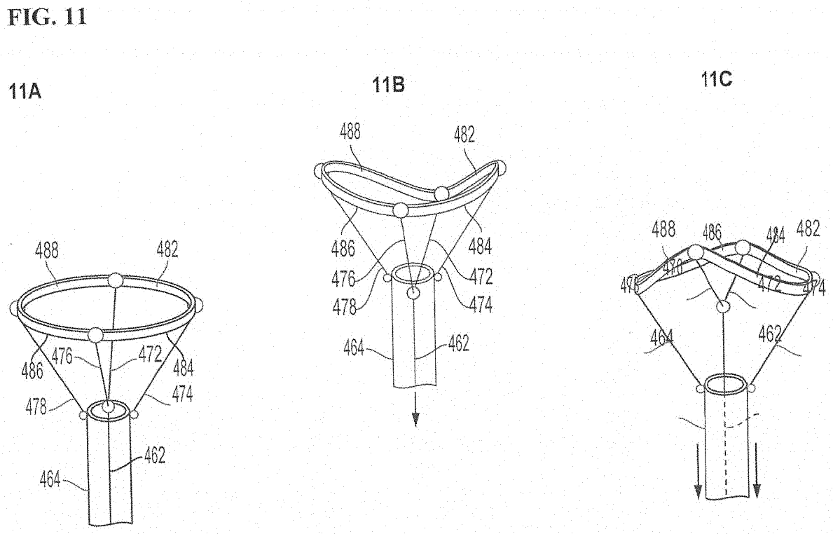

FIG. 11 shows three different aspects or perspectives of the deployed ring-shaped structure's four segments 482, 484, 486, 488 secured by four distal guidewires 472, 474, 476, 478 with a single primary guidewire 462 drawing or releasing the ring-shaped structure and progressively allowing the ring-shaped structure to deform or collapse as progressively shown in FIGS. 11A, 11B and 11C.

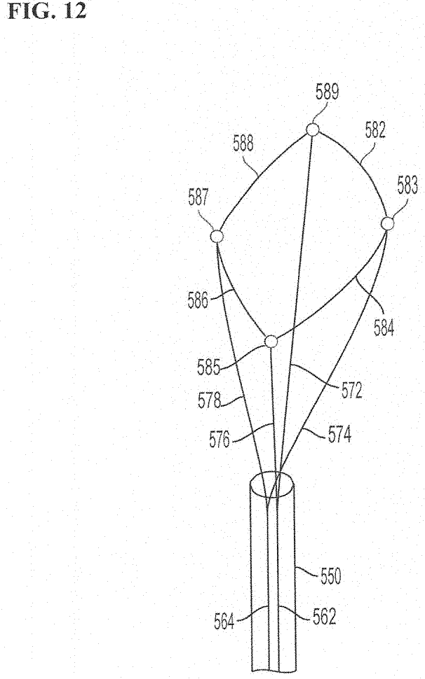

FIG. 12 shows a different perspective of the deployed ring-shaped structure segments 582, 584, 586, 588 with the connection points 583, 585, 587, 589 connecting the segments to the distal guidewires 572, 574, 576, 578 which are in turn connected to primary retraction guidewires 562, 564 within the catheter 550.

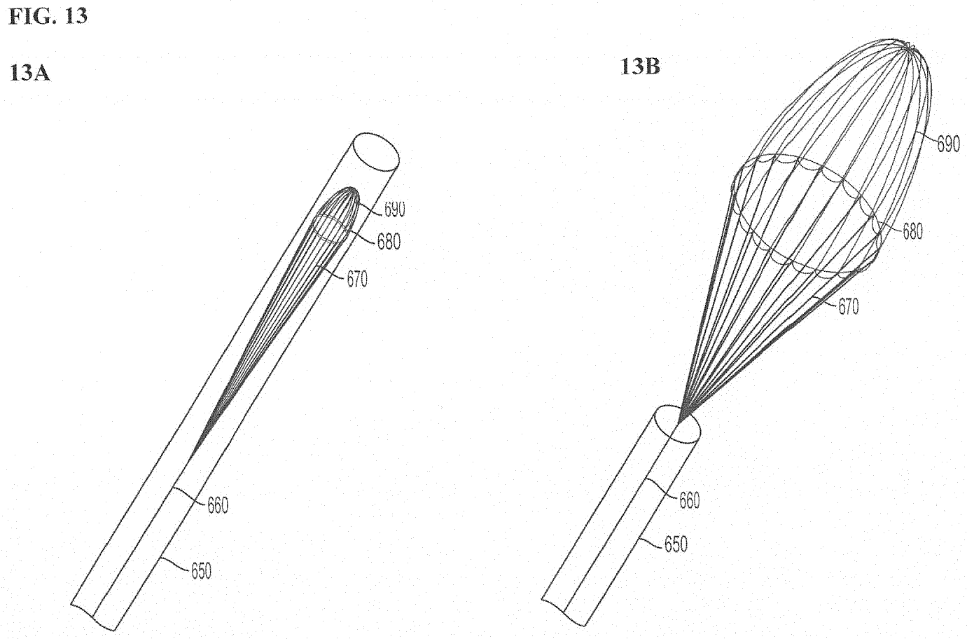

FIG. 13 (13A, 13B) shows a pre-deployed 13A and deployed 13B catheter delivery assembly comprising a catheter 650, single retraction guidewire 660, multiple dual wire element distal retraction guidewires 670, the dual wire element distal retraction guidewires 670 being attached to sides of struts 680 forming the ring-shaped circular opening supporting elements for the wire mesh web 690 (shown without cross wire hatching).

What is claimed is:

1. An intravascular thrombus retraction device comprises wires that are compressible into a compact cylindrical form within a catheter and which are self-expandable into a wire mesh web with at least some parallel wires forming openings in the wire mesh sufficient to allow fluid passage and small enough to filter particles of at least 0.001 mm, a base of the wire mesh web connected to radially ring-shaped structure supporting and maintaining an opening in the base of the wire mesh and forming a thrombus capture volume, the ring-shaped structure being compressible into the catheter and being self-expandable when free of compressive forces within the catheter to open up into the open, expanded ring-shaped structure, maintaining the opening in the opening in the base of the wire mesh, multiple intermediate guide wires are connected to and spaced about the ring-shaped structure, the multiple intermediate guide wires are connected to withdrawal guidewires extending into the catheter, at least some of the wires in the wire mesh having protrusions extending inwardly into the thrombus capture volume, at least some of the protrusions having a height less than a distance between the at least some parallel wires.

2. The device of claim 1 wherein the wire comprises a non-thrombogenic metal and the protrusions have a height of less than 0.001 mm.

3. The device of claim 2 wherein the protrusions comprise elements extending from surfaces of the wires and the protrusions have concave, convex, flat, curvilinear or pointed tips.

4. The device of claim 1 comprising two catheters, a first catheter containing the wire mesh and ring-shaped structure in a compressed, non-expanded state, and a second catheter containing a compressed and expandable collection receptacle, the collection receptacle positioned within the second catheter such that upon release from the catheter, the collection receptacle expands to provide an opening in an opposed position with respect to the opening in the base of the wire mesh of a released and expanded wire mesh and ring-shaped structure.

5. The device of claim 1 wherein the ring-shaped structure comprises or is attached to struts which place expanding or restraining force on the ring-shaped structure to maintain the opening in an expanded and open position.

6. The device of claim 4 wherein the collection receptacle comprises or is attached to struts which place expanding or restraining force on the opening in the opposed position to maintain the opening in the opposed position in an expanded and open position.

7. The device of claim 4 wherein the collection receptacle comprises or is attached to struts which place expanding or restraining force on the opening in the opposed position to maintain the opening in the opposed position in an expanded and open position.

8. A method of capturing a thrombus within vasculature comprising providing an intravascular thrombus retraction device comprised of wires that are compressible into a compact cylindrical form within a catheter and which are self-expandable into a wire mesh web with at least some parallel wires forming openings in the wire mesh sufficient to allow fluid passage and small enough to filter particles of at least 0.001 mm, a base of the wire mesh web connected to radially ring-shaped structure supporting and maintaining an opening in the base of the wire mesh and forming a thrombus capture volume, the ring-shaped structure being compressible into the catheter and being self-expandable when free of compressive forces within the catheter to open up into the open, expanded ring-shaped structure, maintaining the opening in the opening in the base of the wire mesh, multiple intermediate guide wires are connected to and spaced about the ring-shaped structure, the multiple intermediate guide wires are connected to withdrawal guidewires extending into the catheter, at least some of the wires in the wire mesh having protrusions extending inwardly into the thrombus capture volume, at least some of the protrusions having a height less than a distance between the at least some parallel wires;
the method comprising:
a) inserting the device into a blood vessel having a thrombus;
b) advancing a distal end of the catheter of the device towards a thrombus;
c) deploying the compressed wire mesh and ring-shaped structure distally past the thrombus;
d) expanding the wire mesh and ring-shaped structure past the thrombus;
e) retracting the wire mesh and ring-shaped structure by applying tension to the withdrawal guidewires in a first retraction step; and
f) capturing the thrombus within the wire mesh during the first retraction step.

9. The method of claim 8 wherein the wire comprises a non-thrombogenic metal and the protrusions have a height of less than 0.001 mm, and during the first retraction step, the protrusions engage and grasp a surface of the thrombus.

10. A method of treating deep vein thrombosis and pulmonary embolisms comprising accessing a venous vessel of a patient, inserting a retraction catheter containing an intravascular thrombus retraction device comprised of wires that are compressible into a compact cylindrical form within a catheter and which are self-expandable into a wire mesh web with at least some parallel wires forming openings in the wire mesh sufficient to allow fluid passage and small enough to filter particles of at least 0.001 mm, a base of the wire mesh web connected to radially ring-shaped structure supporting and maintaining an opening in the base of the wire mesh and forming a thrombus capture volume, the ring-shaped structure being compressible into the catheter and being self-expandable when free of compressive forces within the catheter to open up into the open, expanded ring-shaped structure, maintaining the opening in the opening in the base of the wire mesh, multiple intermediate guide wires are connected to and spaced about the ring-shaped structure, the multiple intermediate guide wires are connected to withdrawal guidewires extending into the catheter, at least some of the wires in the wire mesh having protrusions extending inwardly into the thrombus capture volume, at least some of the protrusions having a height less than a distance between the at least some parallel wires into the venous circulatory system to a site of clot, inserting an aspiration catheter with wall-mounted suction attached to an inflow port of the aspiration catheter, removing clot and other debris from the venous vessel, and removing both soft and hard components of a vascular obstruction with one pass over a volume that contained both hard components and soft components of the clot and other debris.

11. The method of claim 10, wherein the device is equipped with a collecting mechanism in a collecting catheter configured to pass over the retraction catheter, and the collection catheter deploys a collecting structure while moving the collecting catheter beyond the end of the guiding catheter, the collecting structure surrounding the object when the object is retracted while withdrawing the intravascular thrombus retraction device.

13. The intravascular thrombus retraction device of claim 1 further constituted as a multi-lumen, multi-functional catheter system comprising a plurality of axial lumens, wherein at least one physiological measuring device is present within a clot retraction catheter, and wherein said physiological measuring device is in communication to a host computer configured to receive information to be evaluated by the host computer to provide procedures for deep vein thrombosis and/or pulmonary embolism treatment plans, wherein the host computer stores multiple treatment planning and therapy procedures to be executed by the intravascular thrombus retraction device individual patients, and in response to data from the physiological measuring device the host computer provides an operator of the intravascular thrombus retraction device specific procedures to actively modify an existing treatment plan for the deep vein thrombosis and/or pulmonary embolism treatment plans as therapy progresses.

15. The intravascular thrombus retraction device of claim 10 further characterized as comprising: an elongate flexible catheter body having a proximal end, a distal end and a central lumen extending longitudinally through the catheter body, wherein the catheter comprises a catheter with a variable durometer outer jacket, wherein the catheter wall thickness ratio of the inner diameter to the outer diameter is 0.80 or higher, wherein the tensile strength of the catheter is higher than 2 lbs.

16. The intravascular thrombus retraction device of claim 1 further constituted as comprising an intravascular catheter having a distal end and a proximal end, the catheter having an inner lumen and an outer lumen, wherein an aspiration pump is attached to the proximal end of the catheter, and a mechanically actuated positive displacement powered by a rotating motor, wherein the motor rotates at a speed below 2000 RPM when driving the aspiration pump and wherein the speed of the motor is cycled at a frequency below 10 Hz.
